(19) United States
(12) Patent Application Publication
(10) Pub. No.: US 2007/0193345 A1
(43) Pub. Date: Aug. 23, 2007

(54) Liquid-condition detection sensor

(75) Inventors: Takashi Yamamoto, Aichi (JP); Takeo Sasanuma, Aichi (JP); Yoshikuni Sato, Aichi (JP)

Correspondence Address: SUGHRUE MION, PLLC, 2100 PENNSYLVANIA AVENUE, N.W., SUITE 800, WASHINGTON, DC, 20037, US

(73) Assignee: NGK SPARK PLUG CO., LTD., Nagoya (JP)

(21) Appl. No.: 11/699,550

(22) Filed: Jan. 30, 2007

(30) Foreign Application Priority Data
Jan. 31, 2006 (JP) 2006-023234

(51) Int. Cl. G01N 11/00; G01N 33/00; G01N 33/487

(52) U.S. Cl. 73/61.41; 73/53.01

(57) ABSTRACT

A liquid-condition detection sensor (1) includes a concentration sensor element (260) for detecting the condition of liquid to be measured; a wiring board (40) including a driving control circuit (41) disposed above the concentration sensor element (260); a cable (50) mechanically connected to the wiring board (40), extending downward from the wiring board (40), and establishing an electrical connection between the driving control circuit (41) and the concentration sensor element (260); an inner tube (221) surrounding at least a portion of a cable (50) in a loose condition; and a fixing holder section (94) fixedly holding a portion (51) of the cable (50) located between the wiring board (40) and the concentration sensor element (260).

### BACKGROUND OF THE INVENTION

1. Field of the Invention

The present invention relates to a liquid-condition detection sensor for detecting the condition of liquid to be measured.

2. Description of the Related Art

Exhaust gases from automobiles equipped with a diesel engine or the like contain substances such as nitrogen oxides (NOx). In recent years, various measures have been taken to purify such exhaust gases for the purpose of, particularly, environmental protection and preventing contamination of the living environment. One measure used to purify exhaust gas is an exhaust gas purification apparatus.

The exhaust gas purification apparatus is mounted in the automobile and decomposes harmful nitrogen oxides (NOx) for rendering them harmless by means of an NOx selective catalytic reduction (SCR) system. The NOx selective catalytic reduction (SCR) system uses a urea aqueous solution as a reducing agent. The urea aqueous solution is contained in a tank mounted in the automobile.

In order to effectively decompose nitrogen oxides (NOx), the concentration of the urea aqueous solution (the concentration of urea in the urea aqueous solution) must be maintained within a specified range.

Even when a urea aqueous solution of specified concentration is charged into the tank, the concentration of the urea aqueous solution may fall outside the specified range with the elapse of time or the like. Also, a worker may erroneously mix light oil or water in the tank. In order to cope with these problems, a device for determining the concentration of urea in the urea aqueous solution contained in the tank has been proposed as a sensor for detecting whether or not the urea aqueous solution is within a specified concentration range (Patent Document 1).

The urea-concentration-determining device disclosed in Patent Document 1 includes a concentration-determining sensor section and a support section. The concentration-determining sensor section has a concentration-detecting portion, which includes a heating element and a temperature-sensing element, and a liquid-temperature-detecting portion for measuring the temperature of the urea aqueous solution.

The support section is located at an upper end portion of the urea-concentration-determining device and has an attachment portion for attaching to an opening section of the urea-aqueous-solution tank, and a circuit board located above the attachment portion. A tubular member supports the concentration-determining sensor section located below the attachment portion. The circuit board of the support section has a concentration detection circuit and is covered with a cover member. The circuit board is electrically connected through conductors to the concentration-detecting portion and liquid-temperature-detecting portion of the concentration-determining sensor section. In the urea-concentration-determining device according to Patent Document 1, the conductors which are electrically connected to the circuit board at their one ends extend through the tubular member of the support section such that portions thereof are not held or restrained, and are electrically connected at their other ends to predetermined regions of the concentration-detecting portion and the liquid-temperature-detecting portion.

[Patent Document 1] Japanese Patent Application Laid-Open (kokai) No. 2005-84026

3. Problems to be Solved by the Invention

However, during actual use, for example, in a vehicle, a liquid-condition detection sensor configured like the urea-concentration-determining device according to Patent Document 1 may be subjected to vibration or impact, particularly in a vertical direction. If lead wires extend downward while being mechanically connected to a wiring board by soldering or the like, being subjected to such vibration or impact involves the following risk: the weights of the lead wires and vertically applied vibration or impact can generate a highly repeated stress in or exert a large impact force on mechanical connections between the lead wires and the wiring board.

Thus, fatigue induced by repeated stress can cause time-course occurrence of cracking or rupture in the mechanical connections, or an impact force can cause instantaneous occurrence of such cracking or rupture.

This involves risk of generating noise in an output from a sensor element of the liquid-condition detection sensor or, in an extreme case, risk of breakage of a wire with a resultant failure of the sensor.

### SUMMARY OF THE INVENTION

The present invention has been made in view of the above problems of the prior art, and an object thereof is to provide a liquid-condition detection sensor for detecting the condition of liquid in which, even in the case of being subjected to vibration or impact, damage to a mechanical connection between an electrically conductive path member and a wiring board can be suppressed.

The above object of the invention has been achieved by providing a liquid-condition detection sensor, at least a portion of which is immersed in a liquid to be measured and which is adapted to detect a condition of the liquid to be measured. The liquid-condition detection sensor comprises a sensor element, at least a portion of which is in contact with the liquid to be measured and which is adapted to detect a condition of the liquid to be measured; a wiring board disposed above the sensor element and including a driving control circuit which drives the sensor element and receives a measurement signal indicative of the condition of the liquid to be measured from the sensor element; an electrically conductive path member mechanically connected to the wiring board, extending downward from the wiring board, and establishing electrical communication between the driving control circuit and the sensor element; and a fixing holder section fixedly holding a portion of the electrically conductive path member located between the wiring board and the sensor element.

The liquid-condition detection sensor may further comprise a surrounding tube located below the wiring board and above a lower end of the sensor element, the surrounding tube surrounding the electrically conductive path member in a loose condition.

In the liquid-condition detection sensor of the present invention, a portion of the electrically conductive path member is fixedly held by the fixing holder section.

Thus, even when the sensor is subjected to vibration or impact, generation of repeated stress in or exertion of force such as impact force on the mechanical connections between the wiring board and lead wires can be prevented, which could otherwise result from the weight of at least a portion of the electrically conductive path member located below the fixedly held position, and vibration or impact. Therefore, cracking or rupture in the mechanical connections can be prevented, thereby enabling continuous use of the sensor in good condition.

Examples of liquid-condition detection sensors include a liquid temperature sensor, a liquid concentration sensor, a sensor for determining the type of liquid, and a composite sensor of any of these sensors and another sensor.

No particular limitation is imposed on the electrically conductive path member, so long as the member can establish electrical communication between the driving control circuit and the sensor element and can be mechanically connected to the wiring board. Examples of electrically conductive path members include a covered wire in which a strand is covered with resin such as polyethylene, a lead wire such as an enameled wire, a multicore cable in which a plurality of lead wires are formed into a single cable, and a coaxial cable in which braided wires are arranged coaxially with a core.

The wiring board and the electrically conductive path member are mechanically connected together, for example, by soldering cores of lead wires to the wiring board. Alternatively, the wiring board and the lead wires are connected together via respective terminal members.

Preferably, in the above-mentioned liquid-condition detection sensor, the fixing holder section is located above an upper end of the surrounding tube.

In the liquid-condition detection sensor of the present invention, the fixing holder section is disposed above the upper end of the surrounding tube. The fixing holder section can be provided in the liquid-condition detection sensor so as to be located within the surrounding tube at an appropriate position. However, this may involve difficulty in machining the surrounding tube or in assembly such as insertion of the electrically conductive path member into the surrounding tube. By contrast, providing the fixing holder section above the upper end of the surrounding tube provides a high degree of freedom for the structure of the fixing holder section and facilitates assembly.

Preferably, in either of the above-mentioned liquid-condition detection sensors, the fixing holder section holds the electrically conductive path member with a pull-out strength 10 times or greater than a weight of a portion of the electrically conductive path member located below the portion held by the fixing holder section.

In the liquid-condition detection sensor of the present invention, the fixing holder section having the above-mentioned pull-out strength fixedly holds the electrically conductive path member.

Thus, even when the liquid-condition detection sensor mounted in an automobile or the like is subjected to vibration or impact, the fixing holder section can reliably hold the electrically conductive member. Therefore, the occurrence of a defect such as cracking in the mechanical connection made by soldering or the like between the wiring board and the electrically conductive member can be reliably prevented.

In view of enhancing holding power, preferably, the fixing holder section has a pull-out strength 20 times or greater than the weight of a portion of the electrically conductive path member located below the portion held by the fixing holder section.

Preferably, in any one of the above-mentioned liquid-condition detection sensors, the fixing holder section includes a biting holder portion which bitingly holds and deforms a portion of an outer circumference of the electrically conductive path member in a radially inward direction.

In the liquid-condition detection sensor of the present invention, the fixing holder section includes the biting holder portion. Thus, the fixing holder section bites a portion of the electrically conductive member, thereby reliably holding the electrically conductive member.

No particular limitation is imposed on the biting holder portion, so long as the biting holder portion bitingly holds and deforms a portion of the outer circumference of the electrically conductive path member in a radially inward direction. For example, the biting holder portion may be configured so as to bitingly hold and deform, in a radially inward direction, the circumference of a portion of the electrically conductive path member at a plurality of circumferential positions (e.g., at two or more circumferential or diagonal positions). Alternatively, the biting holder portion may be configured so as to press and deform, in a radially inward direction, the circumference of a portion of the electrically conductive path member in a regular gear-like pattern for bitingly holding the portion-to-be-held.

Preferably, in any one of the above-mentioned liquid-condition detection sensors, the surrounding tube has a cylindrical shape; the electrically conductive path member is a solid, columnar cable including a single or a plurality of lead wires; and a diametral difference between an inside diameter of the surrounding tube and an outside diameter of a portion of the cable located within the surrounding tube is 1.5 mm or less.

In the liquid-condition detection sensor of the present invention, the electrically conductive path member to be disposed within the surrounding tube in a loose condition is a solid, columnar cable having an outside diameter which is 1.5 mm or less smaller than the inside diameter of the surrounding tube.

Accordingly, even when the electrically conductive member radially vibrates within the surrounding tube due to external vibration, the surrounding tube limits the vibration, thereby suppressing influence of the vibration on the fixedly held portion of the electrically conductive path member, and further, on a portion of the electrically conductive path member which is mechanically connected to the wiring board.

Preferably, in any one of the above-mentioned liquid-condition detection sensors, the liquid to be measured is a urea aqueous solution.

A target liquid of the liquid-condition detection sensor of the present invention is a urea aqueous solution. The liquid-condition detection sensor can be used to detect, for example, the temperature and the urea concentration of a urea aqueous solution used in an exhaust gas purification apparatus mounted in an automobile equipped with a diesel engine or the like as mentioned above.

In the case where the liquid-condition detection sensor is used in the exhaust gas purification apparatus of an automobile, even when the sensor is subjected to an external force induced particularly by vertical vibration or impact associated with movement or the like of an automobile, the fixing holder section fixedly holds a portion of the electrically conductive path member. Therefore, the influence of such an external force on the mechanical connections between the lead wires and the wiring board can be suppressed.

### BRIEF DESCRIPTION OF THE DRAWINGS

FIG. 1 is a partially cutaway, sectional view showing the configuration and structure of a liquid-condition detection sensor 1 according to an embodiment of the invention.

FIG. 2 is a vertical sectional view of a base section 10 of the liquid-condition detection sensor 1.

FIG. 3 is a vertical sectional view of the base section 10 of the liquid-condition detection sensor 1 as viewed from a different direction.

FIG. 4 is an exploded perspective view of an inner-tube-and-cable holder section 60.

FIG. 5(a) is a top view showing an insulation plate 90, and FIG. 5(b) is a front view thereof.

FIG. 6 (a) is a perspective view showing a lead-wire holder 110, and FIG. 6(b) is a top view thereof.

FIG. 7 is a vertical sectional view of a base section 410 of a liquid-condition detection sensor 401 according to a modified embodiment.

FIG. 8 is a vertical sectional view of the base section 410 of the liquid-condition detection sensor 401 as viewed from a different direction.

FIG. 9 is an exploded perspective view of a cable holder section 460.

FIG. 10(a) is a top view showing an insulation plate 490, and FIG. 10(b) is a front view thereof.

FIG. 11(a) is a top view showing a cable holder 500, FIG. 11(b) a side view thereof, and FIG. 11(c) is a bottom view thereof.

### DESCRIPTION OF REFERENCE NUMERALS

Reference numerals used to identify various structural features in the drawings include the following.
P: axis (of liquid-condition detection sensor)
1, 401: liquid-condition detection sensor
10, 410: base section
20: body member
40: wiring board
SL: connection region (between lead wire and wiring board)
41: driving control circuit
50: cable (electrically conductive member)
50a: lower portion of cable held by fixing holder portion 94
51: portion of cable fixedly held by fixing holder portion 94
52: lead wire (of cable)
D1: outside diameter (of cable)
60, 460: inner-tube-and-cable holder section
70: electrode support member
80: electrode member
90, 490: insulation plate
94: fixing holder portion
95: biting holder portion (of fixing holder portion)
110: lead-wire holder
120: presser plate
210: sensor section
220: liquid level sensor portion
221: inner tube (surrounding tube)
221u: proximal end (of inner tube)
D2: inside diameter of inner tube
250: liquid concentration sensor portion
260: concentration sensor element
260d: lower end (of concentration sensor element)
500: cable holder
500A: first cable holder (on one side)
500B: second cable holder (on the other side)
505A, 505B: biting holder portion (fixing holder portion)

### DETAILED DESCRIPTION OF THE PREFERRED EMBODIMENTS

A liquid-condition detection sensor according to an embodiment of the present invention will be described with reference to the drawings. However, the present invention should not be construed as being limited thereto.

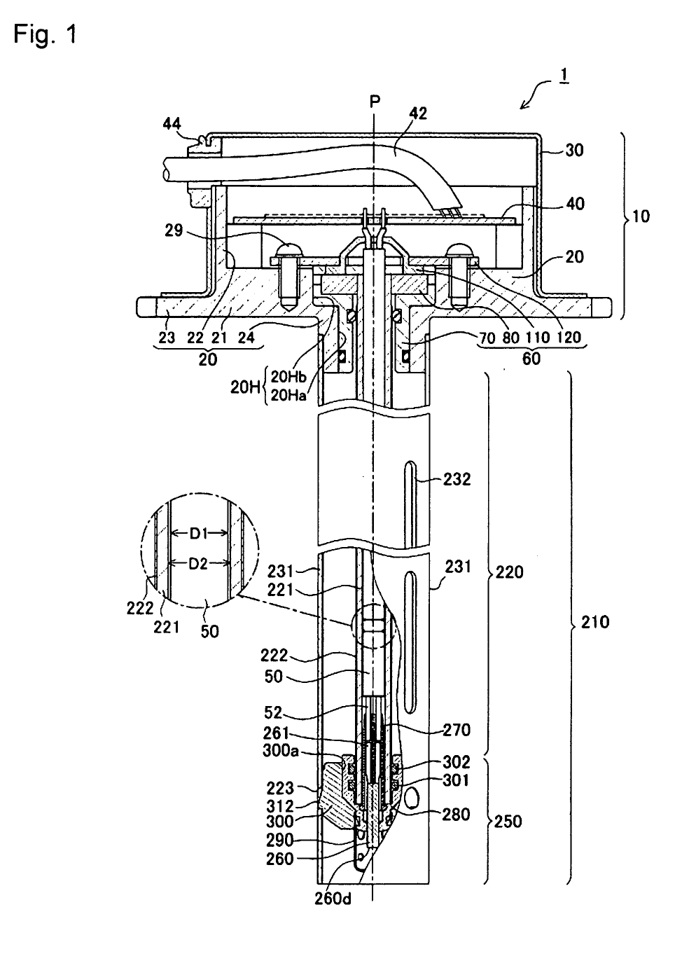

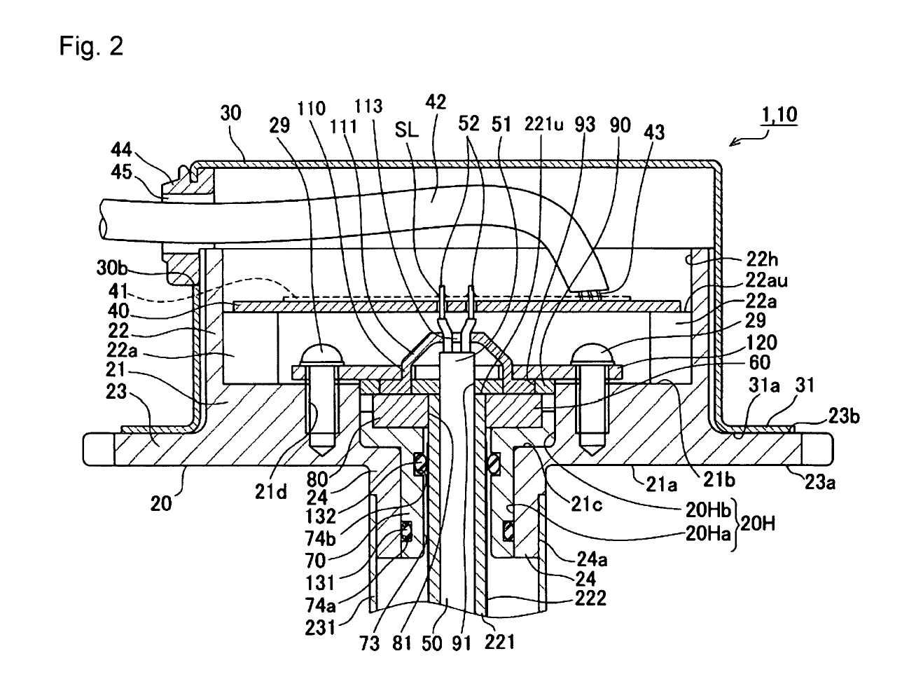

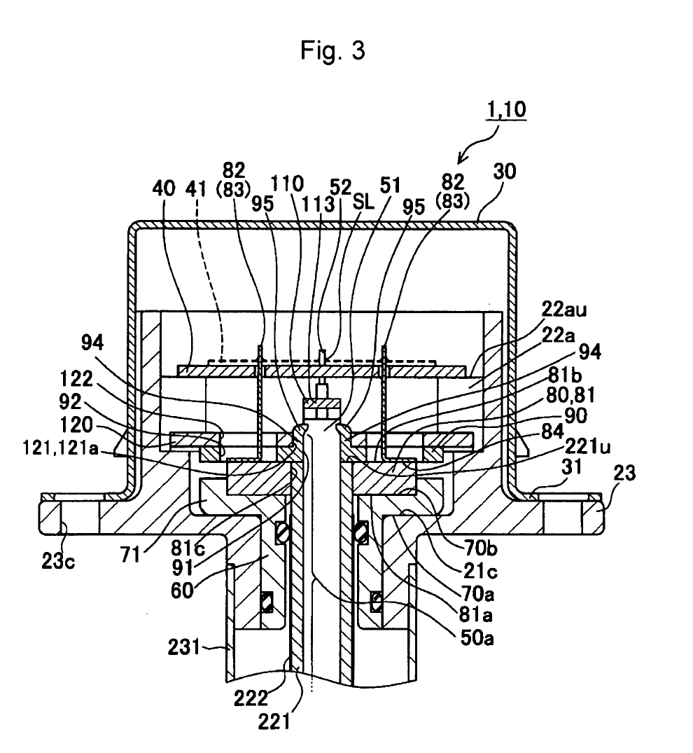

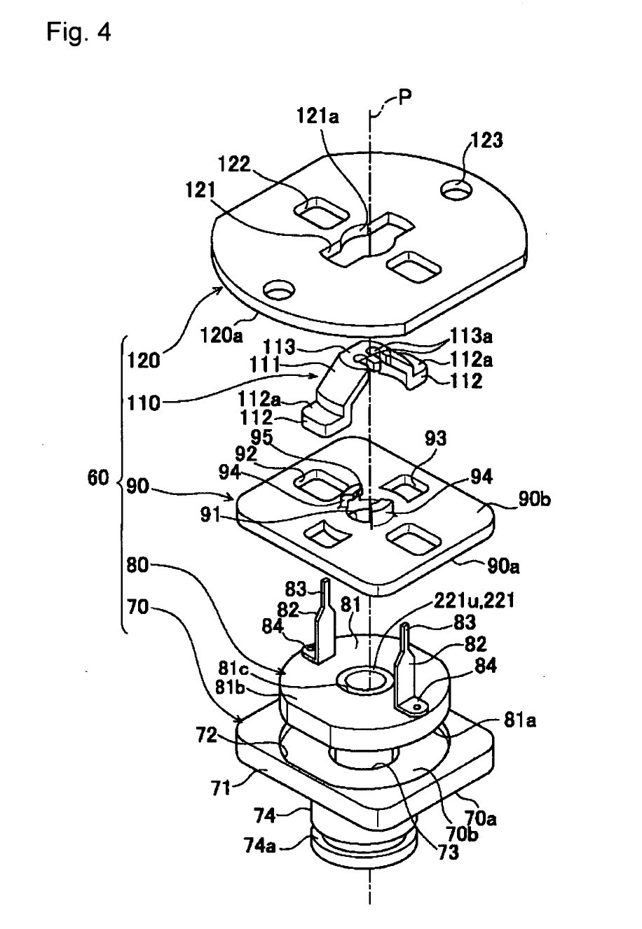

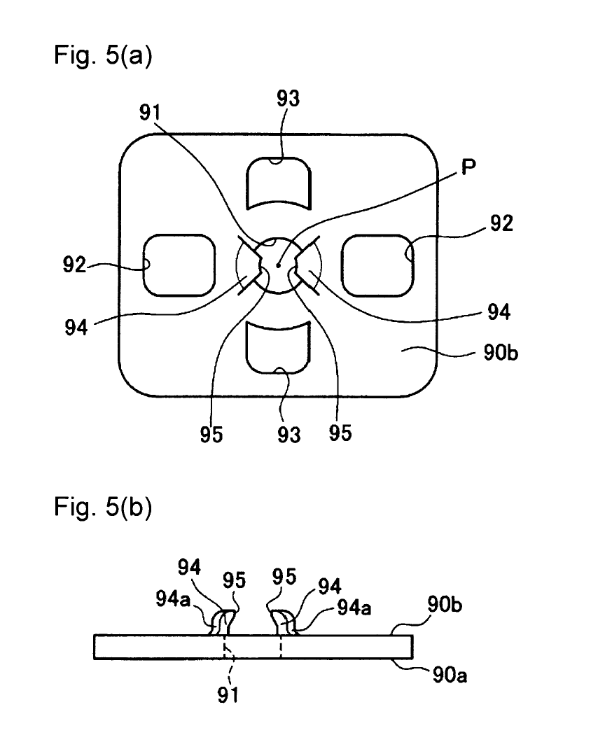

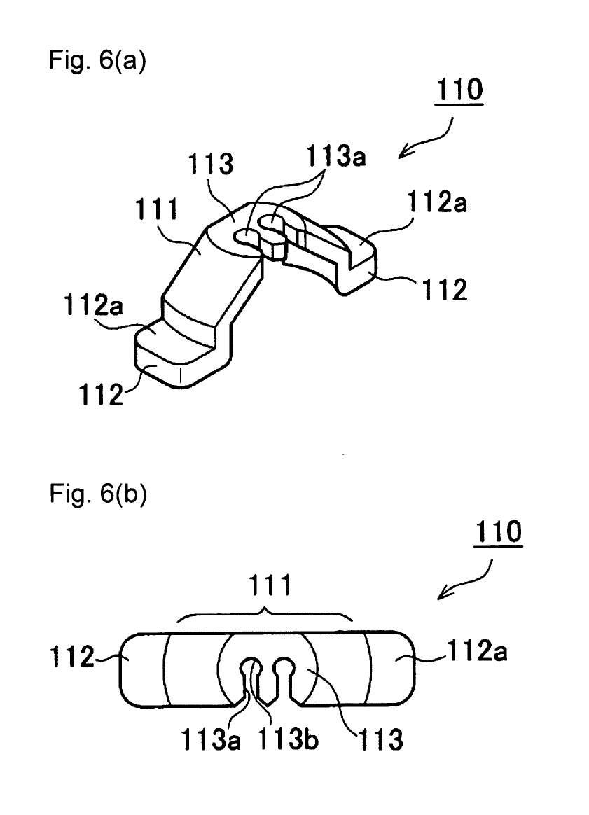

FIG. 1 is a partially cutaway, sectional view showing the configuration and structure of a liquid-condition detection sensor 1 according to an embodiment of the present embodiment. FIG. 2 is a vertical sectional view of a base section 10. FIG. 3 is a vertical sectional view of the base section 10 as viewed from a different direction. FIG. 4 is an exploded perspective view of an inner-tube-and-cable holder section 60. FIG. 5 is a pair of views showing an insulation plate 90, wherein FIG. 5(a) is a top view, and FIG. 5(b) is a front view. FIG. 6 includes a pair of views showing a lead-wire holder 110, wherein FIG. 6(a) is a perspective view, and FIG. 6(b) is a top view.

In the description of the liquid-condition detection sensor 1 according to the present embodiment as well as components thereof, the upper side along the direction of an axis P (axial direction) in FIG. 1 is called the proximal-end side, and the lower side in FIG. 1 is called the distal-end side.

The liquid-condition detection sensor 1 according to the present embodiment is used to detect, for example, the concentration and liquid level of a urea aqueous solution contained in a tank of an exhaust gas purification apparatus. Such gas purification apparatus is adapted for rendering harmless nitrogen oxides (NOx) contained in exhaust gas from the automobile equipped with a diesel engine or the like by reducing the nitrogen oxides with the urea aqueous solution.

The liquid-condition detection sensor 1 includes the base section 10 located at the proximal-end side thereof and a tubular sensor section 210 extending toward the distal-end side from the base section 10.

The sensor section 210 has a liquid level sensor portion 220 and a liquid concentration sensor portion 250 located at the distal end of the liquid level sensor portion 220. The base section 10 has a body member 20; a cover member 30; a wiring board 40 covered with the body member 20 and the cover member 30; a cable 50 which connects the wiring board 40 and the liquid concentration sensor portion 250; and an inner-tube-and-cable holder section 60 for holding, in the body member 20, an inner tube 221 of the liquid level sensor portion 220, as well as the cable 50.

In use of the liquid-condition detection sensor 1, the base section 10 is attached to a tank (not shown) containing the urea aqueous solution, and the sensor section 210 provided on the distal-end side of the base section 10 is immersed in the urea aqueous solution.

First, the base section 10 of the liquid-condition detection sensor 1 will be described.

The body member 20 of the base section 10 is formed from metal and includes, as shown in FIG. 1, a body portion 21 assuming the form of a substantially rectangular plate, a surrounding portion 22 assuming the form of a rectangularly tubular wall and extending toward the proximal-end side from a peripheral portion of the body portion 21, a flange portion 23 projecting radially outward from the side surface of the body portion 21, and a cylindrical outer-tube connection portion 24 projecting toward the distal-end side from the center of the body portion 21.

As shown in FIGS. 2 and 3, the body member 20 has, at the center, a cable insertion hole 20H extending along the axis P through the body portion 21 and through the outer-tube connection portion 24. The cable insertion hole 20H includes a circular hole portion 20Ha having a circular cross section and located toward the distal-end side, and a square hole portion 20Hb located on the proximal-end side of the circular hole portion 20Ha, having a substantially square cross section, and having sides longer than the diameter of the circular hole portion 20Ha. A shoulder surface 21c is formed in the body portion 21 between the circular hole portion 20Ha and the square hole portion 20Hb. Members used to form the inner-tube-and-cable holder section 60 are disposed and held in the cable insertion hole 20H.

The body portion 21 has two tapped holes 21d in a bottom surface 21b located toward the proximal-end side; the tapped holes 21d are diagonally located with the cable insertion hole 20H therebetween; and screws for fixing a presser plate 120, described below, are screwed into the respective tapped holes 21d.

The surrounding portion 22 has a rectangularly tubular shape. A board accommodation hole 22h is a rectangular-parallelepiped-shaped internal space of the surrounding portion 22. While assuming the form of a closed-bottomed hole whose bottom surface is the bottom surface 21b, which is located toward the proximal-end side, of the body portion 21, the board accommodation hole 22h communicates, at its central portion, with the cable insertion hole 20H of the body portion 22. Four board support portions 22a project into the board accommodation hole 22h from four respective corners of the surrounding portion 22. The wiring board 40 is held on board support surfaces 22au, which are located toward the proximal-end side, of the board support portions 22a such that four corners thereof abut and are screwed (not shown) onto the respective board support surfaces 22au. As for position along the axis P, the board support surfaces 22au (lower surface of the wiring board 40) are located toward the proximal-end side in relation to an arch portion 111 (lead-wire-holding portion 113), described below, of the lead-wire holder 110 of the inner-tube-and-cable holder section 60. Thus, the board support portions 22a support the wiring board 40 at a position which is located away from the bottom surface 21b located toward the proximal-end side.

The flange portion 23 has a flange-seating surface 23a flush with a tank attachment surface 21a, located toward the distal-end side, of the rectangular body portion 21 and has, as viewed in plane, a rectangular, annular flange shape extending radially outward (left-and-right direction in FIGS. 2 and 3). The flange portion 23 has bolt insertion holes 23c (see FIG. 3). By means of the bolt insertion holes 23c, the liquid-condition detection sensor 1 is attached to an unillustrated tank such that the flange-seating surface 23a faces the periphery of an opening portion of the tank. A cover-member abutment surface 23b opposite the flange-seating surface 23a abuts a body-member abutment surface 31a of a flange portion 31 of the cover member 30, which will be described later.

A distal end portion of the outer-tube connection portion 24 is formed into a mating stepped portion 24a smaller in diameter than a proximal end portion of the outer-tube connection portion 24. An outer tube 231, which partially constitutes the liquid level sensor portion 220, is fitted onto the mating stepped portion 24a, and the outer tube 231 and the mating stepped portion 24a are fixed together by welding or a like bonding method. The body member 20 is electrically connected to a pattern having the ground potential of a driving control circuit 41 formed on the wiring board 40, whereby the outer tube 231 electrically communicates with the driving control circuit 41 on the wiring board 40 via the body member 20 including the mating stepped portion 24a and thus is at ground potential.

Next, the wiring board 40, which is a member of the base section 10, will be described. The wiring board 40 assumes the form of a rectangular, flat plate. Although unillustrated in detail, the driving control circuit 41 is formed on the wiring board 40. The driving control circuit 41 includes a CPU, a ROM, a RAM, and other electronic circuits, and drives the liquid level sensor portion 220 and the liquid concentration sensor portion 250 by use of power supplied through an external connection cable 42 and also processes output signals therefrom. The driving control circuit 41 is also configured so as to output the results of processing to an unillustrated external electronic circuit (e.g., ECU) through the external connection cable 42.

In operation of the liquid level sensor portion 220, the driving control circuit 41 applies an AC voltage across the inner tube 221 and the outer tube 231, described below, to thereby detect the magnitude of capacitance therebetween; calculates the liquid level of urea aqueous solution on the basis of the detected capacitance; and sends an output signal indicative of the liquid level to an external circuit.

In operation of the liquid concentration sensor portion 250, the driving control circuit 41 also supplies current to an unillustrated heat-generating resistor of a concentration sensor element 260 immersed in the urea aqueous solution for a predetermined time through the cable 50 to thereby heat the concentration sensor element 260; detects a variation in voltage (electric potential) between opposite ends of the heat-generating resistor associated with the flow of current therethrough over a predetermined time; calculates the concentration of the urea aqueous solution; and sends an output signal indicative of the concentration to an external circuit.

The external connection cable 42 is connected to the driving control circuit 41 by soldering one (first) ends of lead wires 43 to respectively predetermined regions of the wiring board 40.

As mentioned previously, the wiring board 40 is disposed within the board accommodation hole 22h of the surrounding portion 22 and is covered for protection with the cover member 30 having a section resembling a squarish letter U. The cover member 30 assumes the form of a closed-bottomed rectangular tube and has a flange portion 31 at the periphery of an open end. The cover member 30 has a grommet hole 30b formed in its side portion (left side surface in FIG. 2). A rubber grommet 44 is fitted into the grommet hole 30b. The external connection cable 42 is loosely inserted through an insertion hole 45 of the grommet 44. While the body-member abutment surface 31a of the flange portion 31 abuts the cover-member abutment surface 23b of the flange portion 23 of the body member 20, the cover member 30 covers the wiring board 40 and the outer surface of the surrounding portion 22, thereby protecting the wiring board 40 and the like from external effects. Although unillustrated, the interior of the surrounding portion 22 of the base section 10 is filled with a urethane resin so as to waterproof the interior of the surrounding portion 22 in which the wiring board 40 and the like are accommodated.

Next, the cable 50 will be described. The cable 50 establishes electrical communication between the driving control circuit 41 and the concentration sensor element 260 and is mechanically connected to the wiring board 40. The cable 50 is a solid, columnar two-core cable internally including two lead wires 52 and an insulating coat covering the lead wires 52. The cable 50 is loosely inserted through the inner tube 221, described below. A diametral difference ΔD (D2−D1) between the inside diameter D2 (7.0 mm) of the inner tube 221 and the outside diameter D1 (6.4 mm) of the cable 50 is 0.6 mm. By adjusting the diameters such that the diametral difference AD becomes 1.5 mm or less, radial movement of the cable 50 is suppressed within the inner tube 221.

Next, the inner-tube-and-cable holder section 60 will be described with reference to FIGS. 2 to 6.

As shown in the exploded perspective view of FIG. 4, the inner-tube-and-cable holder section 60 of the present embodiment includes five members. Specifically, the members are, from the distal-end side (the lower side in FIG. 4) to the proximal-end side (the upper side in FIG. 4), an electrode support member 70, an electrode member 80, the insulation plate 90, the lead-wire holder 110, and the presser plate 120.

The inner-tube-and-cable holder section 60 including the above members mechanically holds the inner tube 221, which serves as an electrode of the liquid level sensor portion 220 of the sensor section 210, and is electrically connected to the driving control circuit 41 on the wiring board 40 via the electrode member 80. Also, the inner-tube-and-cable holder section 60 holds the cable 50, which electrically connects the concentration sensor element 260, described below, and the driving control circuit 41 on the wiring board 40, in a hanging manner by means of fixing holder portions 94 of the insulation plate 90.

The members of the inner-tube-and-cable holder section 60 will be sequentially described. First, the electrode support member 70 is formed from an electrically insulative, hard resin (e.g., nylon) and has a support flange portion 71 located toward the proximal-end side and assuming the form of a square plate, and a cylindrical inner-tube-surrounding portion 74 extending toward the distal-end side from a body-member abutment surface 70a, which is the distal end surface of the support flange portion 71. The support flange portion 71 has an electrode-member accommodation recess 72 formed on the surface located toward the proximal-end side. The electrode-member accommodation recess 72 assumes a disk-like form with two truncations so as to receive the electrode member 80 (electrode substrate 81), described next. The bottom surface of the electrode-member accommodation recess 72 is an electrode-member abutment surface 70b, and the electrode member 80 (electrode substrate 81) abuts the accommodation recess 72.

The inner-tube-surrounding portion 74 has an inner-tube insertion hole 73 extending therethrough. The inner-tube insertion hole 73 communicates with the electrode-member accommodation recess 72 of the support flange portion 71. The inner tube 221 is inserted into the inner-tube insertion hole 73.

As shown in FIGS. 1 to 3, the electrode support member 70 is disposed in the cable insertion hole 20H of the body member 20. Specifically, the inner-tube-surrounding portion 74 is disposed in the circular hole portion 20Ha of the cable insertion hole 20H; i.e., in the outer-tube connection portion 24, and the support flange portion 71 is disposed in the square hole portion 20Hb; i.e., in the body portion 21. Accordingly, the body-member abutment surface 70a of the support flange portion 71 and the shoulder surface 21c of the body portion 21 abut one another.

As shown in FIGS. 2 to 4, the outer circumference of the inner-tube-surrounding portion 74 has an outer O-ring embedment groove 74a. An outer O ring 131 is disposed in the outer O-ring embedment groove 74a, thereby providing a liquid-tight seal between the body member 20 (outer-tube connection portion 24) and the electrode support member 70 (inner-tube-surrounding portion 74).

Furthermore, the inner circumference of the inner-tube-surrounding portion 74 has an inner O-ring embedment groove 74b. An inner O ring 132 is disposed in the inner O-ring embedment groove 74b, thereby providing a liquid-tight seal between the inner tube 221 (more specifically, an insulating film 222 on the outer circumference of the inner tube 221) and the electrode support member 70 (inner-tube-surrounding portion 74).

Next, the electrode member 80 of the inner-tube-and-cable holder section 60 will be described. As easily understood from FIG. 4, the electrode member 80 of metal includes the electrode substrate 81 assuming the form of an annular plate with two truncations on the outer side surface, and two electrode terminals 82 fixedly attached to the electrode substrate 81. The electrode substrate 81 has an inner-tube insertion hole 81c at the center. The diameter of the inner-tube insertion hole 81c is such that the inner tube 221 can be fitted thereinto, and extends through the electrode substrate 81 between an electrode-terminal connection surface 81b located toward the proximal-end side and a support-member abutment surface 81a located toward the distal-end side.

A proximal end portion of the inner tube 221 is fitted into the inner-tube insertion hole 81c of the electrode substrate 81, and the electrode substrate 81 and the inner tube 221 are welded together. The surface of a proximal end 221u (end surface) of the inner tube 221 is flush with the electrode-terminal connection surface 81b of the electrode substrate 81.

As described below, the insulating film 222 is formed on the outer surface of the inner tube 221. However, the insulating film 222 is not formed on a portion of the inner tube 221 which is present in the inner-tube insertion hole 81c of the electrode substrate 81 (a proximal end portion of the inner tube 221). Accordingly, the inner tube 221 and the electrode substrate 81 are in direct and electrical contact with one another.

Electrode-fixing portions 84 of the two electrode terminals 82 are arranged symmetrically with respect to the inner-tube insertion hole 81c on the electrode-terminal connection surface 81b of the electrode substrate 81 and are mechanically fixed and electrically connected to the electrode substrate 81 by spot welding. The electrode terminal 82 is an L-shaped terminal member formed by bending at a right angle between a board insertion portion 83 and the electrode-fixing portion 84.

The present embodiment employs two electrode terminals 82. At least a single electrode terminal will suffice. However, in order to reliably connect the inner tube 221 and the driving control circuit 41 on the wiring board 40 with low electrical resistance, the present embodiment employs two electrode terminals. More than two electrode terminals may be employed to connect the driving control circuit 41 and the inner tube 221.

The electrode member 80 is positioned such that a portion of the electrode substrate 81 is fitted into the electrode-member accommodation recess 72 of the electrode support member 70, and such that the electrode-member abutment surface 70b and the support-member abutment surface 81a abut one another.

The board insertion portions 83 of the electrode terminals 82 extend through respective electrode-terminal insertion holes 92 of the insulation plate 90 and respective electrode-terminal insertion holes 122 of the presser plate 120, described below, and further extend through the wiring board 40 and are electrically connected to the driving control circuit 41 by soldering (see FIGS. 3 and 4).

Next, the insulation plate 90 of the inner-tube-and-cable holder section 60 will be described. The insulation plate 90 is formed from an electrically insulative, hard resin and, as understood from FIGS. 4 and 5, assumes the form of a rectangular plate. The insulation plate 90 has, in its central portion, a cable insertion hole 91 extending therethrough between an electrode-member abutment surface 90a located toward the distal-end side and a presser-plate abutment surface 90b located toward the proximal-end side. The two fixing holder portions 94 are formed on a portion of the presser-plate abutment surface 90b around the cable insertion hole 91 in opposition to each other with respect to the cable insertion hole 91, and each assumes the form of a projecting claw which stands toward the proximal-end side (upward in FIG. 4).

The fixing holder portions 94 arcuately extend toward the proximal-end side (upward in FIG. 4) from a portion of the presser-plate abutment surface 90b around the cable insertion hole 91 and have respective outer peripheral surfaces 94a, which are portions of a substantially cylindrical surface. End portions of the fixing holder portions 94 are bent in a radially inward direction. Accordingly, the end portions of the fixing holder portions 94 serve as a pair of biting holder portions 95 which presses and deforms in a radially inward direction a portion-to-be-held 51 of the cable 50 inserted through the cable insertion hole 91, to thereby bitingly hold a protruding portion (the portion-to-be-held 51) of the insulating coat of the cable 50 located between the wiring board 40 and the inner tube (surrounding tube) 221 (see FIG. 3). According to the present embodiment, a pair of biting holder portions 95 of the fixing holder portions 94 bitingly holds the portion-to-be-held 51 of the cable 50, so that the cable 50 can be reliably held with sufficient holding power.

According to the present embodiment, while being located toward the proximal-end side (upward in FIGS. 2 and 3) in relation to the proximal end 221u of the inner tube 221, the fixing holder portions 94 (biting holder portions 95) fixedly hold the portion-to-be-held 51 of the cable 50. That is, in the present embodiment, the inner-tube-and-cable holder section 60 is configured such that a portion of the cable 50 which is located toward the proximal-end side in relation to the proximal end 211u of the inner tube 211; i.e., a portion of the cable 50 around which the inner tube 211 is absent, serves as the portion-to-be-held 51 to be fixedly held thereby. Accordingly, as compared with the case where a fixing holder section for fixedly holding the cable 50 is formed within the inner tube 211, the inner-tube-and-cable holder section 60 can be readily formed and facilitates application of holding power.

As described above, the electrode-terminal insertion holes 92 which allow the respective board insertion portions 83 of the electrode terminals 82 to extend therethrough are formed in the insulation plate 90 outside the respective fixing holder portions 94. Two holder reception holes 93 are formed in the insulation plate 90 in opposition to each other with respect to the cable insertion hole 91, and are located radially outward of the cable insertion hole 91 and 90 degrees about the axis P away from the fixing holder portions 94 and the electrode-terminal insertion holes 92. Base portions 112 of the lead-wire holder 110, described next, are disposed in the respective holder reception holes 93, whereby the lead-wire holder 110 is positioned.

The insulation plate 90 is positioned along the axis P in such manner that the electrode-member abutment surface 90a abuts the electrode-terminal abutment surface 81b of the electrode member 80 (electrode substrate 81). The insulation plate 90 is circumferentially positioned so as to be fitted into the square hole portion 20Hb of the cable insertion hole 20H of the body member 20.

Next, the lead-wire holder 110 of the inner-tube-and-cable holder section 60 will be described. The lead-wire holder 110 is formed of an electrically insulative resin and includes, as shown in FIGS. 4 and 6, the two base portions 112 and the arch portion 111, which connects the base portions 112 and projects arcuately toward the proximal-end side. A top portion of the arch portion 111 serves as the lead-wire-holding portion 113 whose proximal end surface is flat. The lead-wire-holding portion 113 has two cutouts 113a into which the two respective lead wires 52 of the cable 50 can be laterally fitted and which are spaced apart from one another. A deep portion of each of the cutouts 113a serves as a lead-wire-holding region 113b which is slightly greater in diameter than an entrance portion of the cutout 113a. The lead-wire-holding regions 113b hold the respective lead wires 52.

The base portions 112 of the lead-wire holder 110 have the same thickness as that of the insulation plate 90. As described above, the base portions 112 are disposed in the respective holder reception holes 93, thereby positioning the lead-wire holder 110. Proximal end surfaces 112a of the base portions 112 abut a pressing surface 120a of the presser plate 120, described next, to thereby be pressed and fixed by the presser plate 120.

As shown in FIGS. 2 and 3, the arch portion 111 is disposed so as to project toward the proximal-end side (upward in FIGS. 2 and 3) from the presser-plate abutment surface 90b, which is located toward the proximal-end side, of the insulation plate 90. The lead-wire-holding portion 113, which is located toward the proximal-end side in relation to the fixing holder portions 94 of the insulation plate 90, holds the lead wires 52 of the cable 50. As such, the lead wires 52 are fitted into the respective lead-wire-holding regions 113b of the cutouts 113a to thereby be individually held and separated so as to insulate the lead wires from one another. As shown in FIGS. 2 and 3, the lead wires 52 are inserted through the wiring board 40 and are electrically and mechanically connected, in connection regions SL by soldering or the like, to the driving control circuit 41 formed on the wiring board 40.

The method of connecting the lead wires 52 and the driving control circuit 41 is not limited to soldering. For example, they may be connected via various kinds of terminal members.

Next, the presser plate 120 of the inner-tube-and-cable holder section 60 will be described. The presser plate 120 is formed from metal and assumes a disk-like form with two truncations on the outer side surface. The presser plate 120 has, at its central portion, a holder insertion hole 121 which has a shape resembling an elongated rectangle and whose long sides are arcuately swollen at their central portions to thereby form a fixing-holder-portions-surrounding region 121a. The presser plate 120 has the two electrode-terminal insertion holes 122, which are located radially outward of the fixing-holder-portions-surrounding region 121a and in opposition to each other with respect to the fixing-holder-portions-surrounding region 121a. Furthermore, the presser plate 120 has two set-screw insertion holes 123, which are located radially outward of the holder insertion hole 121, 90 degrees about the axis P away from the fixing-holder-portions-surrounding region 121a and the electrode-terminal insertion holes 122, and in opposition to each other with respect to the holder insertion hole 121.

The holder insertion hole 121 allows insertion therethrough of the arch portion 111 of the lead-wire holder 110 and the fixing holder portions 94 (biting holder portions 95) of the insulation plate 90. The fixing-holder-portions-surrounding region 121a of the holder insertion hole 121 is fitted to the outer peripheral surfaces 94a of the fixing holder portions 94 of the insulation plate 90, thereby pressing the fixing holder portions 94 in a radially inward direction. This increases holding power with which the fixing holder portions 94 (biting holder portions 95) bitingly hold (fixedly hold) the portion-to-be-held 51 of the cable 50.

Furthermore, in the case where the fixing holder portions 94 (biting holder portions 95) continue bitingly holding (fixedly holding) the portion-to-be-held 51 of the cable 50, there is some risk of time-course reduction in holding power. This is because of the radially outward movement of the fixing holder portions 94 (biting holder portions 95) caused by reaction force against the pressing force which presses the portion-to-be-held. However, according to the present embodiment, the fixing-holder-portions-surrounding region 121a of the holder insertion hole 121 presses the fixing holder portions 94 of the insulation plate 90 in a radially inward direction, thereby preventing time-course reduction in holding power for bitingly holding (fixedly holding) the portion-to-be-held 51 of the cable 50. Accordingly, holding power for bitingly holding (fixedly holding) the portion-to-be-held 51 of the cable 50 by means of the fixing holder portions 94 (biting holder portions 95) can be maintained over a long period of time.

As described above, the board insertion portions 83 of the electrode terminals 82 are inserted through the respective electrode-terminal insertion holes 122 of the presser plate 120.

Presser-plate set screws 29 are inserted through the respective set-screw insertion holes 123 and are screwed into the respective tapped holes 21d formed in the body portion 21 of the body member 20, whereby the presser plate 120 is fixed to the body portion 21 of the body member 20 while being urged toward the distal-end side (downward in FIGS. 2 and 3).

Accordingly, the pressing surface 120a of the presser plate 120 presses, toward the distal-end side, the base portions 112 of the lead-wire holder 110 via the proximal end surfaces 112a of the base portions 112, and the insulation plate 90 via the presser-plate abutment surface 90b, whereby the base portions 112 and the insulation plate 90 are held between the presser plate 120 and the electrode substrate 81 of the electrode member 80. The electrode substrate 81 presses the electrode-member abutment surface 70b of the electrode support member 70 via the support-member abutment surface 81a thereof. Furthermore, the electrode support member 70 presses the shoulder surface 21c of the body portion 21 of the body member 20 via the body-member abutment surface 70a thereof.

Thus, the component members of the inner-tube-and-cable holder section 60 are fixed within the cable insertion hole 20H. The inner tube 221 is also fixed. Furthermore, the cable 50 is fixedly held at its portion-to-be-held 51 by the inner-tube-and-cable holder section 60; specifically, by the biting holder portions 95 of the fixing holder portions 94 of the insulation plate 90.

Next, the sensor section 210 will be described. First, the liquid level sensor portion 220 of the sensor section 210 will be described.

As shown in FIG. 1, the liquid level sensor portion 220 includes the outer tube 231 extending along the axis P (the axial direction) and having a cylindrical shape, and the inner tube 221 disposed coaxially within the outer tube 231 and having a cylindrical shape. The outer tube 231 and the inner tube 221 are spaced a predetermined distance apart from one another. The inner tube 221 of the present embodiment corresponds to the surrounding tube of the invention.

The inner tube 221 of the liquid level sensor portion 220 is formed from metal and faces the outer tube 231 while being electrically insulated from the outer tube 231, so as to serve as one of two electrodes for detecting a liquid level. As discussed above, the inner tube 221 electrically communicates with the driving control circuit 41 via the inner-tube-and-cable holder section 60 (electrode member 80). In order to ensure electrical insulation from the outer tube 231, the outer circumferential surface of the inner tube 221 is covered with the insulating film 222, which is formed from, for example, a fluorine-containing resin such as PTFE, PFA, or ETFE, an epoxy resin, or a polyimide resin.

As discussed above, the inner tube 221 is inserted into the inner-tube-surrounding portion 74 of the electrode support member 70 and into the inner-tube insertion hole 81c of the electrode substrate 81 in the inner-tube-and-cable holder section 60, and is fixedly attached by welding or the like to the electrode substrate 81 such that the surface of the proximal end 221u is flush with the electrode-terminal connection surface 81b.

The outer tube 231 is also formed from metal; serves as the other electrode for detecting a liquid level; and electrically connects to the driving control circuit 41 so as to be at ground potential. The outer tube 231 has a plurality of narrow slits 232 whose longitudinal direction coincides with the direction of the axis P and which are located at predetermined positions, whereby the urea aqueous solution (liquid to be measured) can be accommodated in a space between the outer tube 231 and the inner tube 221 while communicating with the exterior of the outer tube 231 via the slits 232. The distal end of the outer tube 231 is opened, and the proximal end of the outer tube 231 is welded to the outer-tube connection portion 24 of the body member 20. A rubber bushing 300, described below, intervenes between a distal end portion of the outer tube 231 and a distal end portion of the inner tube 221. The outer tube 231 has a plurality of engagement holes 223 which are engaged with respective projections 312 of the rubber bushing 300 and are circumferentially arranged at equally spaced predetermined positions (see FIG. 1).

The principle of detecting the liquid level of the urea aqueous solution by means of the liquid level sensor portion 220 will now be described. The liquid level sensor portion 220 is immersed in the urea aqueous solution so as to introduce the urea aqueous solution into the space between the outer tube 231 and the inner tube 221 (insulating film 222) through the slits 232 and the like. Then, the space between the outer tube 231 and the inner tube 221 is divided into a region where the urea aqueous solution is present, and a region where the urea aqueous solution is absent, in accordance with liquid level. When AC voltage is applied between the inner tube 221 and the outer tube 231, AC current corresponding to capacitance generated therebetween flows. The capacitance between the inner tube 221 and the outer tube 231 varies with liquid level; thus, the AC current varies with liquid level. Accordingly, the liquid level of the urea aqueous solution can be detected from the magnitude of the capacitance (AC current).

Next, the liquid concentration sensor portion 250 will be described with reference to FIG. 1.

The liquid concentration sensor portion 250 is located at the distal end of the liquid level sensor portion 220 and includes the concentration sensor element 260, a separator 270, a holder member 280, a protector 290, and the rubber bushing 300.

The concentration sensor element 260 is held in the interior of the holder member 280 such that a portion thereof projects toward the distal-end side (downward in FIG. 1) from the holder member 280. A pair of connection terminals 261 is connected to the proximal end of the concentration sensor element 260 while projecting toward the proximal-end side. The lead wires 52 of the cable 50 are soldered to the respective connection terminals 261. Thus, the concentration sensor element 260 is electrically connected to the driving control circuit 41 on the wiring board 40 via the connection terminals 261 and the cable 50.

The inner tube 221 is inserted between the concentration sensor element 260 and the holder member 280 from the proximal-end side. Accordingly, a proximal end portion of the concentration sensor element 260 and the connection terminals 261 are located in the interior of a distal end portion of the inner tube 221. As discussed above, the cable 50 extends through the inner tube 221. Two O rings 301 and 302 are disposed between the outer circumferential surface of the inner tube 221 (insulating film 222) and the inner circumferential surface of the holder member 280 so as to prevent entry of the urea aqueous solution (liquid to be measured) into the inner tube 221 through a clearance therebetween. The inner tube 221 is located toward the distal-end side in relation to the wiring board 40 (below the wiring board 40 in FIG. 1) and toward the proximal-end side in relation to a lower end 260d of the concentration sensor element 260 (above the lower end 260d in FIG. 1).

Next, the principle of detecting the urea concentration of the urea aqueous solution by means of the liquid concentration sensor portion 250 (specifically, the concentration sensor element 260) will briefly be described. First, it is known that the thermal conductivity of the urea aqueous solution varies depending on the concentration of urea contained in the urea aqueous solution. Thus, when the urea aqueous solution present around the concentration sensor element 260 is heated for a certain time by use of a heat-generating resistor provided in the concentration sensor element 260, the rate of temperature rise of the urea aqueous solution varies with the concentration of the urea aqueous solution. It is also known that, when a constant current is passed through the heat-generating resistor, the resistance of the heat-generating resistor varies substantially proportionally to a rise in temperature around the heat-generating resistor. Thus, the urea concentration of the urea aqueous solution can be detected by heating the concentration sensor element 260, and specifically, by passing a constant current through the heat-generating resistor provided in the concentration sensor element 260 for a certain time period and detecting a change in voltage (electric potential) developed between the opposite ends of the heat-generating resistor associated with a change in resistance of the heat-generating resistor as measured between start and end of current supply to the resistor.

The separator 270 is fitted into a distal end portion of the inner tube 221. The separator 270 is formed from an electrically insulative, rubberlike elastic material. In the inner tube 221, the separator 270 accommodates therein a proximal end portion of the concentration sensor element 260 and the connection terminals 261, and intervenes between the inner tube 221 and the connection terminals 261 and between the connection terminals 261 so as to insulate these members from one another.

The protector 290 is fitted to a distal end portion of the holder member 280. The protector 290 covers and protects a portion of the concentration sensor element 260 which projects toward the distal-end side from the holder member 280. The protector 290 has an appropriate number of liquid communication holes at appropriate positions for allowing the urea aqueous solution to flow between the interior and the exterior thereof.

The rubber bushing 300 has a holder-holding hole 300a whose shape fits the geometry of the holder member 280. While holding the holder member 280 therein, the rubber bushing 300 is fixedly held in a distal end portion of the outer tube 231 with the projections 312 being engaged with respective engagement holes 223 of the outer tube 231. In this manner, the liquid concentration sensor portion 250 is held between a distal end portion of the inner tube 221 and that of the outer tube 231.

Next, the load associated with the cable 50 will be described. As discussed above, the cable 50 is connected at its distal end (at its lower end in FIG. 1) to the connection terminals 261 of the concentration sensor element 260. The cable 50 is bitingly held at its portion-to-be-held 51, which is its proximal end portion (its upper end portion in FIGS. 1, 2, and 3), by the biting holder portions 95 of the fixing holder portions 94 of the insulation plate 90 of the inner-tube-and-cable holder section 60. The lead wires 52 are exposed at the proximal end of the cable 50 and are electrically and mechanically connected, in the connection regions SL by soldering or the like, to the driving control circuit 41 on the wiring board 40.

The cable 50 is loosely inserted into the inner tube 221.

Accordingly, if the insulation plate 90 does not have the fixing holder portions 94 (biting holder portions 95), and thus the cable 50 is not fixedly held, the weight of the cable 50 is exerted through the lead wires 52 on the connection regions SL where the lead wires 52 are connected to the wiring board 40 (driving control circuit 41). Furthermore, since the liquid-condition detection sensor 1 of the present embodiment, together with a urea aqueous solution tank, is mounted in a vehicle or the like, the liquid-condition detection sensor 1 is subjected to vibration or impact in the course of operation of the vehicle or the like. Thus, in addition to the weight of the cable 50, load associated with such vibration or impact; particularly, load associated with vibration or impact along the axis P, is exerted on the connection regions SL. Therefore, repeated exertion of vibration involves risk of time-course occurrence of the following problem in the connection regions SL, or exertion of a large impact involves risk of instantaneous occurrence of the following problem in the connection regions SL: cracking in solder, breakage of the lead wire(s) 52, or detachment of the lead wire(s) 52 from the wiring board 40.

However, in the liquid-condition detection sensor 1 of the present embodiment, as shown in FIG. 3, not only are the lead wires 52 soldered to the wiring board 40, but also the insulation board 90 has the fixing holder portions 94 (biting holder portions 95), which bitingly hold (fixedly hold) the portion-to-be-held 51 of the cable 50.

Specifically, in the liquid-condition detection sensor 1 of the present embodiment, the fixing holder portions 94 (biting holder portions 95) of the insulation plate 90 have a holding power (of holding the cable 50) 10 times or greater than the weight of a lower portion 50a of the cable 50 (approx. 50 gf=approx. 0.49 N) as measured in terms of pull-out strength; specifically, 20N.

Accordingly, the fixing holder portions 94 (biting holder portions 95) can support the weight of the lower portion 50a (see FIG. 3), which is located toward the distal-end side (downward) from the portion-to-be-held 51. Even when the liquid-condition detection sensor 1 of the present embodiment is subjected to vibration or impact, the fixing holder portions 94 (biting holder portions 95) can also support the associated load. Thus, exertion of an excessively large load on the connection regions SL is prevented, where the wiring board 40 and the lead wires 52 are connected. Thus, the liquid-condition detection sensor 1 is free from the occurrence of defects such as cracking in solder in the connection regions SL, breakage of the lead wire(s) 52, or detachment of the lead wire(s) 52 from the wiring board 40 and therefore can continuously be used in an appropriate condition.

The cable 50 pull-out strength of the liquid-condition detection sensor 1 is defined as follows. The inner tube 221, the outer tube 231, and the liquid concentration sensor 250 are removed from the liquid-condition detection sensor 1. By use of a universal strength tester, the body member 20 is fixed, and a distal end portion of the cable 50 is pulled along the axis P at a speed of 100 mm/min. Tensile strength as measured when the cable 50 is detached from the fixing holder portions 94 (biting holder portions 95) is taken as the pull-out strength (holding power).

When external vibration causes lateral vibration of the cable 50, there is risk of generating stress associated with this vibration (lateral vibration) in the biting holder portions 95 (portion-to-be-held 51) and in the connection regions SL where the wiring board 40 and the lead wires 52 are connected.

However, in the liquid-condition detection sensor 1 of the present embodiment, as discussed above, the diametral difference ΔD (=D2−D1) between the inside diameter D2 (7.0 mm) of the inner tube 221 and the outside diameter D1 (6.4 mm) of the cable 50 is 0.6 mm. By employing a small diametral difference ΔD; specifically, a ΔD of 1.5 mm or less, even when the cable 50 vibrates radially within the inner tube 221 due to subjection to external vibration, the inner tube 221 restricts the vibration (see FIG. 1). Accordingly, the influence of such vibration of the cable 50 on the portion-to-be-held 51 and on the connection regions SL where the wiring board 40 and the lead wires 52 are connected can be suppressed.

### Modified Embodiment

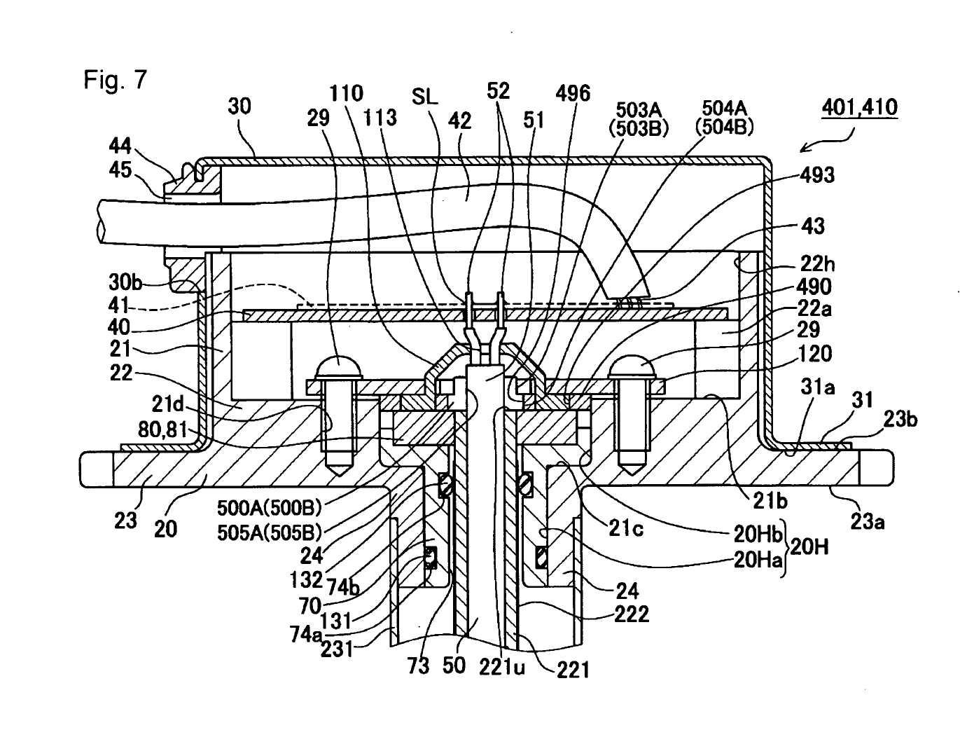

Next, a modified embodiment of the above-described embodiment will be described with reference to FIGS. 7 to 11.

In the above-described embodiment, the inner-tube-and-cable holder section 60 of the base section 10 is configured such that the fixing holder portions 94 (biting holder portions 95) for fixedly holding the cable 50 are formed integrally with the insulation plate 90, and such that the biting holder portions 95 of the fixing holder portions 94 hold the cable 50 by biting the portion-to-be-held 51 of the cable 50.

In contrast, an inner-tube-and-cable holder section 460 according to the modified embodiment differs from the above-described embodiment in that a cable holder 500 for fixedly holding the cable 50 is formed separately from an insulation plate 490. The remaining features of the modified embodiment are similar to those of the above-described embodiment.

Accordingly, the following description will focus on features different from those of the above-described embodiment. Description of similar features is omitted or simplified. In the drawings, similar members or portions are denoted by reference numerals similar to those of the above-described embodiment.

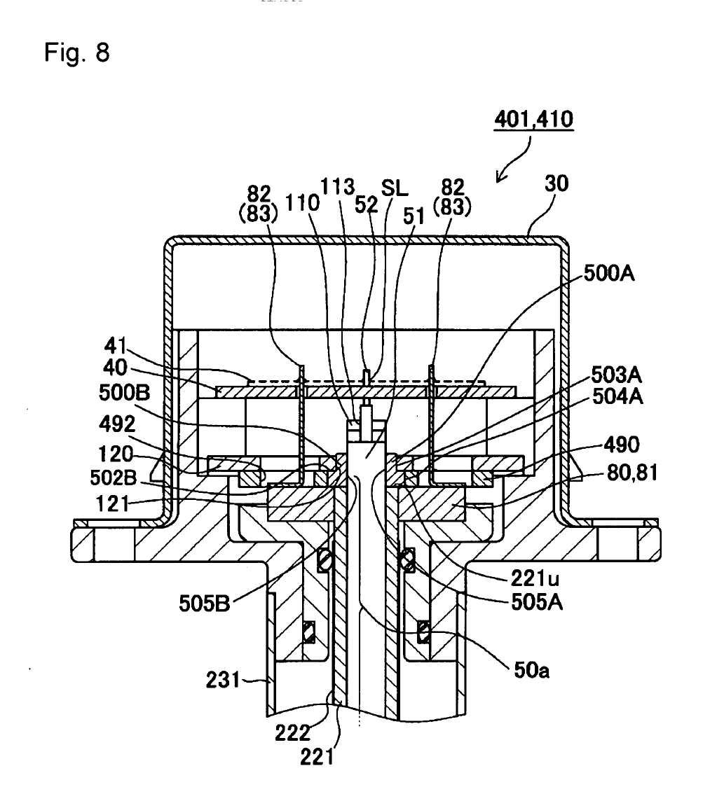

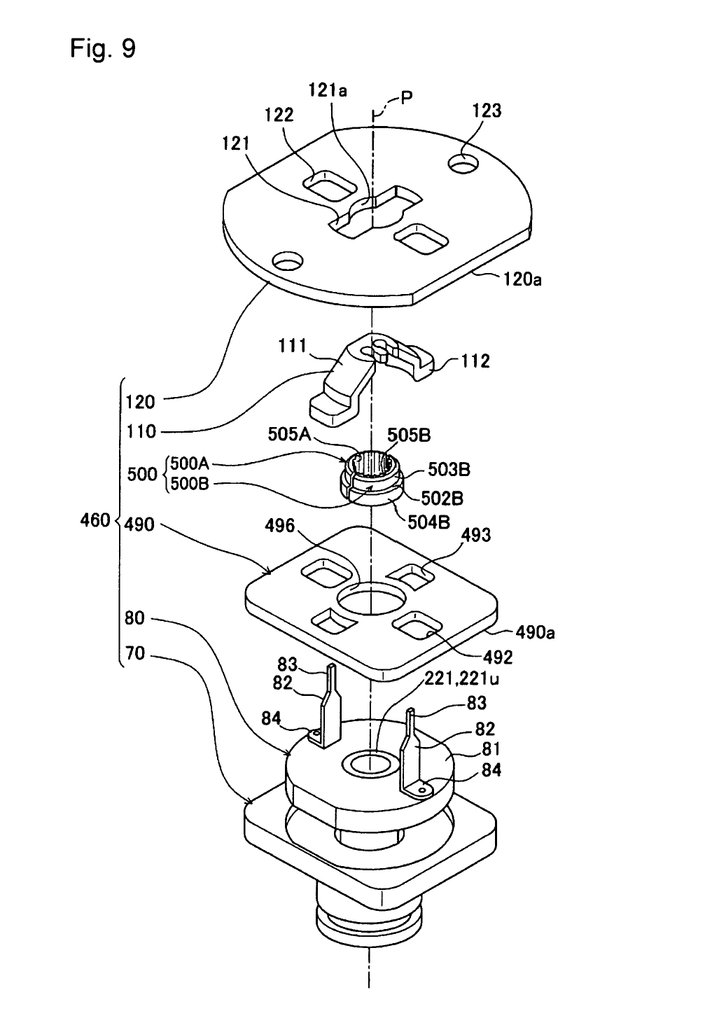

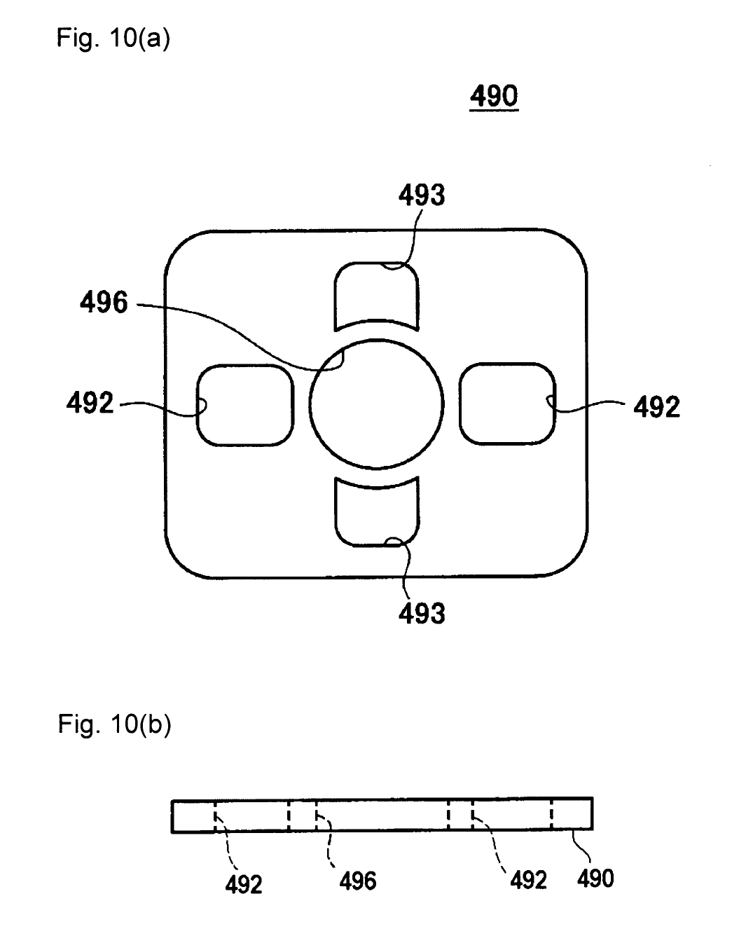

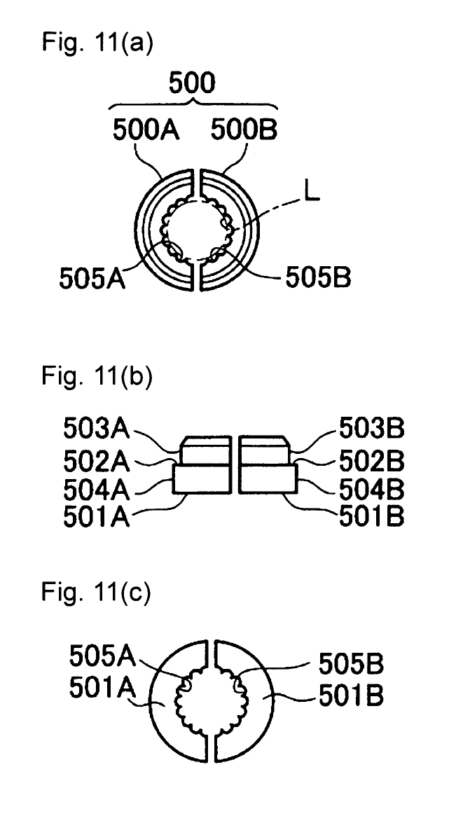

FIG. 7 is a vertical sectional view of a base section 410 of a liquid-condition detection sensor 401 according to the present modified embodiment. FIG. 8 is a vertical sectional view of the base section 410 of the liquid-condition detection sensor 401 as viewed from a direction perpendicular to FIG. 7. FIG. 9 is an exploded perspective view of the inner-tube-and-cable holder section 460. FIG. 10 is a pair of views showing the insulation plate 490, wherein FIG. 10(a) is a top view, and FIG. 10(b) is a front view. FIG. 11 is a series of views showing a cable holder 500, wherein FIG. 11(a) is a top view, FIG. 11(b) is a side view, and FIG. 11(c) is a bottom view.

As shown in FIG. 9, the inner-tube-and-cable holder section 460 in the present modified embodiment includes the electrode support member 70, the electrode member 80, the lead-wire holder 110, and the presser plate 120, which are similar to those of the above-described embodiment, as well as the insulation plate 490 and the cable holder 500, which are employed instead of the insulation plate 90 in the above-described embodiment.

The insulation plate 490 is formed from an electrically insulative, hard resin. As shown in FIGS. 9 and 10, the insulation plate 490 assumes the form of a rectangular plate and has, in its central portion, a holder insertion hole 496 for allowing insertion of the cable holder 500 thereinto. The insulation plate 490 further has electrode-terminal insertion holes 492 and holder reception holes 493, which are similar to the electrode-terminal insertion holes 92 and holder reception holes 93, respectively, of the insulation plate 90 of the above-described embodiment.

As shown in FIGS. 9 and 11, the cable holder 500 includes two members; i.e., a first cable holder 500A and a second cable holder 500B each having a shape resembling the letter C. The first cable holder 500A and the second cable holder 500B are formed from en electrically insulative, hard resin, and, as shown in FIGS. 9 and 11, face each other so as to form a shape resembling a stepped cylinder. The first cable holder 500A and the second cable holder 500B assume substantially the same form. The first cable holder 500A and the second cable holder 500B have a shape such that disposition-in-insulation-plate portions 504A and 504B each assuming the form of a large-diameter arc and disposition-in-presser-plate portions 503A and 503B each assuming the form of a small-diameter arc are superposed, respectively.

The disposition-in-insulation-plate portions 504A and 504B are disposed in the above-mentioned holder insertion hole 496 of the insulation plate 490. The disposition-in-presser-plate portions 503A and 503B are fitted into the holder insertion hole 121 of the presser plate 120 (see FIG. 9) such that the outer circumferential surfaces thereof abut the fixing-holder-portions-surrounding region 121a of the holder insertion hole 121 to thereby be pressed in a radially inward direction.

Presser-plate abutment surfaces 502A and 502B, which are shoulder surfaces between the disposition-in-insulation-plate portions 504A and 504B and the disposition-in-presser-plate portions 503A and 503B, respectively, are pressed by the presser plate 120; specifically, by the pressing surface 120a of the presser plate 120.

Electrode-member abutment surfaces 501A and 501B, which are distal end surfaces of the first cable holder 500A and the second cable holder 500B, respectively, abut the electrode-terminal connection surface 81b of the electrode substrate 81 and the proximal end 221u of the inner tube 221.

As shown in FIG. 11(a), the inner portions of the first cable holder 500A and the second cable holder 500B serve as biting holder portions 505A and 505B, respectively, each assuming the form of alternate recesses and projections circumferentially arranged in a gear-like fashion and extending along the axis P. When the first cable holder 500A and the second cable holder 500B are disposed in opposition to each other, the diameter of an imaginary circle L inscribed in the biting holder portions 505A and 505B is slightly smaller than the outside diameter D1 of the cable 50.

As shown in FIGS. 7 and 8, in the inner-tube-and-cable holder section 460 of the liquid-condition detection sensor 401 of the present modified embodiment, while the cable 50 is inserted through the holder insertion hole 496 of the insulation plate 490, the first cable holder 500A and the second cable holder 500B are fitted into the holder insertion hole 496 of the insulation plate 490 while facing each other. Furthermore, as in the case of the above-described embodiment, the base portions 112 of the lead-wire holder 110 are disposed in the respective holder reception holes 493, and the arch portion 111 of the lead-wire holder 110 and the disposition-in-presser-plate portions 503A and 503B of the first cable holder 500A and the second cable holder 500B, respectively, are fitted into the holder insertion hole 121 of the presser plate 120. In this procedure, the fixing-holder-portions-surrounding region 121a of the holder insertion hole 121 of the presser plate 120 presses in a radially inward direction the outer circumferential surfaces of the disposition-in-presser-plate portions 503A and 503B of the first cable holder 500A and the second cable holder 500B, respectively. Accordingly, a plurality of elongated projections of the biting holder portions 505A and 505B of the first cable holder 500A and the second cable holder 500B, respectively, press and deform in a radially inward direction corresponding portions of the circumference of the portion-to-be-held 51 of the cable 50, thereby holding the portion-to-be-held 51 of the cable 50 with high holding power (pull-out strength).

Thus, even when the liquid-condition detection sensor 401 of the present embodiment is subjected to vibration or impact as a result of being mounted in an automobile or the like, the inner-tube-and-cable holder section 460; specifically, the biting holder portions 505A and 505B of the first cable holder 500A and the second cable holder 500B, respectively, reliably hold the cable 50. Accordingly, the biting holder portions 505A and 505B support the weight of the lower portion 50a (see FIG. 8) of the cable 50, which lower portion 50a is located toward the distal-end side (downward) from the portion-to-be-held 51, as well as the load associated with vibration or impact. Therefore, the occurrence of a defect is reliably prevented such as cracking in solder in the connection regions SL where the wiring board 40 and the lead wires 52 of the cable 50 are connected, breakage of the lead wire(s) 52, or detachment of the lead wire(s) 52 from the wiring board 40. Thus, the liquid-condition detection sensor 401 can continuously be used in good condition.

Also, in the present modified embodiment, the fixing-holder-portions-surrounding region 121a of the holder insertion hole 121 of the presser plate 120 presses in a radially inward direction the disposition-in-presser-plate portions 503A and 503B of the first and second cable holders 500A and 500B, respectively, thereby preventing time-course reduction in holding power for bitingly holding (fixedly holding) the portion-to-be-held 51 of the cable 50. Accordingly, holding power for bitingly holding (fixedly holding) the portion-to-be-held 51 of the cable 50 by means of the biting holder portions 505A and 505B can be maintained over a long period of time.

Also, in the present modified embodiment, while being located toward the proximal-end side (upward in FIGS. 7 and 8) in relation to the proximal end 221u of the inner tube 221, the biting holder portions 505A and 505B fixedly hold the portion-to-be-held 51 of the cable 50. That is, also, in the present modified embodiment, the inner-tube-and-cable holder section 460 is configured such that a portion of the cable 50 which is located toward the proximal-end side in relation to the proximal end 221u of the inner tube 221 serves as the portion-to-be-held 51 to be fixedly held thereby. Accordingly, as compared with the case where a fixing holder section for fixedly holding the cable 50 is formed within the inner tube 221, the inner-tube-and-cable holder section 460 can be readily formed and facilitates application of holding power.

While the present invention has been described with reference to the above embodiment and the modified embodiment, the present invention is not limited thereto, but may be modified as appropriate without departing from the spirit and scope of the invention.

The above embodiment and modified embodiment are described in reference to the portion-to-be-held 51 which is located near the proximal end (upper end) of the cable 50. However, for example, depending on the shape of the base section 10, the position of the wiring board 40, etc., a portion of an electrically conductive path member such as a cable which is located at an appropriate position between a wiring board and a sensor element may serve as a portion-to-be-held. Even in this case, fixing holder portions (biting holder portions) support the portion-to-be-held, whereby the weight of a lower portion located below the portion-to-be-held and load exerted on the lower portion in association with vibration or impact can be prevented from being exerted on a mechanical connection between the electrically conductive path member and the wiring board. Accordingly, the occurrence of damage to the mechanical connection between the electrically conductive path member and the wiring board can be suppressed.

The above embodiment and modified embodiment employ the cable 50 including the two lead wires 52. However, two lead wires may be independently inserted into the inner tube 221. The greater the weight of the lower portion located below the portion-to-be-held, the higher the effect of the present invention in suppressing damage to the mechanical connection between the electrically conductive path member and the wiring board. Thus, in the case where a cable is used, application of the present invention is further preferred.

This application is based on Japanese Patent Application No. JP 2006-023234 filed Jan. 31, 2006, incorporated herein by reference in its entirety.

What is claimed is:

1. A liquid-condition detection sensor comprising:
a sensor element, at least a portion of which is in contact with the liquid to be measured and which is adapted to detect a condition of the liquid to be measured;
a wiring board disposed above the sensor element and including a driving control circuit which drives the sensor element and receives a measurement signal indicative of the condition of the liquid to be measured from the sensor element;
an electrically conductive path member mechanically connected to the wiring board, extending downward from the wiring board, and establishing electrical communication between the driving control circuit and the sensor element; and
a fixing holder section fixedly holding a portion of the electrically conductive path member located between the wiring board and the sensor element.

2. The liquid-condition detection sensor according to claim 1, further comprising a surrounding tube located below the wiring board and above a lower end of the sensor element, said surrounding tube surrounding the electrically conductive path member in a loose condition.

3. The liquid-condition detection sensor according to claim 2, wherein the fixing holder section is located above an upper end of the surrounding tube.

4. The liquid-condition detection sensor according to claim 1, wherein the fixing holder section holds the electrically conductive path member with a pull-out strength ten times or more greater than a weight of a portion of the electrically conductive path member located below the portion held by the fixing holder section.

5. The liquid-condition detection sensor according to claim 1, wherein the fixing holder section includes a biting holder portion which bitingly holds and deforms a portion of an outer circumference of the electrically conductive path member in a radially inward direction.

6. The liquid-condition detection sensor according to claim 2, wherein the surrounding tube has a cylindrical shape;
the electrically conductive path member comprises a solid, columnar cable including a single or a plurality of lead wires; and
a diametral difference between an inside diameter of the surrounding tube and an outside diameter of a portion of the cable located within the surrounding tube is 1.5 mm or less.

7. The liquid-condition detection sensor according to claim 1, wherein the liquid to be measured is a urea aqueous solution.

8. The liquid-condition detection sensor according to claim 1, wherein the sensor element includes a heat generating resistor having a resistance which changes with a change in temperature, and the driving control circuit inputs an input signal to the heat generating resistor so as to drive the sensor element and receive the measurement signal outputted from the heat generating resistor.

9. The liquid-condition detection sensor according to claim 2, further comprising:
an outer tube located below the wiring board and surrounding the surrounding tube; and
a level sensor portion,
wherein the level sensor portion has a capacitance which changes depending on a level of the liquid to be measured between the surrounding tube and the outer tube.

10. A liquid-condition detection sensor comprising:
a sensor element, at least a portion of which is in contact with a liquid to be measured and which is adapted to detect the condition of the liquid to be measured;
a wiring board including a driving control circuit which drives the sensor;
a cable including a lead wire and an insulating coat covering the lead wire, the lead wire electrically connecting the sensor element to the driving control circuit;
a surrounding tube surrounding the cable in a loose condition; and
a fixing holder section fixedly holding a protruding portion of the insulating coat located between the wiring board and the surrounding tube.
